(19) United States
(12) Patent Application Publication
(10) Pub. No.: US 2018/0064527 A1
(43) Pub. Date: Mar. 8, 2018

(54) Modeling Blood-Brain Barrier in Vitro

(71) Applicant: The Government of the United States of America, as represented by the Secretary of the Navy, Arlington, VA (US)

(72) Inventors: André A. Adams, Burke, VA (US); Kyle A. DiVito, Burkittsville, MD (US); Stella H. North, Alexandria, VA (US); Michael A. Daniele, Raleigh, NC (US)

(21) Appl. No.: 15/697,607

(22) Filed: Sep. 7, 2017

Related U.S. Application Data
(60) Provisional application No. 62/384,312, filed on Sep. 7, 2016.

(51) Int. Cl. A61F 2/06; C12N 5/0793

(52) U.S. Cl. CPC A61F 2/062; C12N 5/0619; C12N 2533/54; C12N 2533/30; C12N 2533/80; C12N 2533/90; C12N 2533/52

(57) ABSTRACT

Synthetic human blood vessels can be constructed using human brain derived endothelial cells and incorporated into a tissue model that contains astrocytes and other neurons and microglia. Multi-cell type microvessels incorporate cell types such as astrocytes and pericytes in order to construct a highly representative blood-brain barrier in vitro model with a functional lumen containing brain-derived microvascular endothelial cells and a polymer wall containing human astrocytes and/or pericytes.

### CROSS-REFERENCE TO RELATED APPLICATIONS

This Application claims the benefit of U.S. Provisional Application 62/384,312 filed on Sep. 7, 2017, the entirety of which is incorporated herein by reference.

### BACKGROUND

The blood-brain barrier (BBB) tightly controls access to crucial activities orchestrated by the central nervous system and is one of the more intricate mechanisms in human biology. At least five different cell types along with various extracellular matrix components help establish BBB function. Arguably the most important functional unit of the BBB relies on a specialized type of endothelial cell, termed the brain-derived microvascular endothelial cell (BMEC) (FIG. 1). These endothelial cells line the lumen of venules, arterioles and tiny capillaries (<25 um outer diameter) present in the human brain and themselves express specialized junctional proteins called tight junctions which provide barrier function. These endothelial cells are critical elements that limit the type and amount of material attempting to gain access to the brain. Excluding the brain from harmful chemicals and restricted molecules is imperative under most circumstances.

However, the barrier provided to the brain by microvascular endothelial cells is the same barrier that has created difficulty for researchers in treating neurodegeneration and cancer-related diseases, as most pharmaceuticals are restricted from gaining access to the brain. In addition to BMECs, astrocytes are neural cells that reside in close proximity to the BMEC-laden capillary. Astrocytes provide the linkage between the capillary and other neurons which interpret the response to stimuli. Another cell type called the pericyte, which like astrocytes, are in close proximity to the BMEC-capillary and are considered glial cells as they act in a supporting role by regulating blood vessel permeability, controlling angiogenesis, cerebral blood flow and neuroinflammation (Herland, van der Meer et al. 2016; Sweeney, Ayyadurai et al. 2016). Other neurons present interpret signals sent from astrocytes and execute excitatory processes in order to propagate the message. Microglial cells play an immune-surveillance role by monitoring the brain for bacterial or viral infection. Brain-derived microvascular endothelial cells, astrocytes, pericytes, neighboring neurons, and microglia are collectively referred to as the neurovascular unit (NVU) (FIG. 1). Non-cellular material such as the various neural basement membranes (BM) also play critical roles in regulating BBB function by modulating direct cell-cell interaction. For example, there are two known basement membranes; the endothelial-associated BM which separates endothelial cells from pericytes; and the parenchymal-associated BM which separates astrocytes from endothelial cells. As an example of their intricacy these BM are indistinguishable from one another under the light microscope, yet are composed of different laminin isoforms, play critical roles in structural support and act as natural ligands to entrap soluble factors released from astrocytes and pericytes which can stimulate tight junction rigidity (Banerjee, Shi et al. 2016).

A critical cellular component of the BBB is the BMEC, which is responsible for generating the endothelial tight junction. Tight junctions (TJ) are linkages between neighboring cells of the same cell type, which ultimately form an extremely restrictive barrier (FIG. 2). Blood vessels found elsewhere throughout the body are largely linked by adherens junctions (AJ) which provide a vital, though more permissive barrier when compared to the BBB. As their name implies, TJs form virtually impermeable linkages between neighboring cells. Unlike endothelial cells present other organs such as the kidney, brain endothelial cells lack fenestrations (transcellular membranous cavities) and contain a limited number of pinocytotic vesicles, both of which are responsible for internalizing ions, solutes and other larger soluble molecules (Satchell and Braet 2009). Endothelial cells of the neural lineage physically link themselves to other neighboring endothelial cells through integral proteins such as claudin, occludin, and zona occludin-1 (ZO-1) which comprise the TJ (FIG. 2). Functionally these proteins are responsible for providing an extremely prohibitive barrier, barring molecules >8 angstroms or approximately 450 Da; by comparison the length of a carbon-carbon bond is approximately 1.5 angstroms, therefore the BBB is a tightly monitored organ system. (Abbott, Patabendige et al. 2010). While BMEC are responsible for the restrictive barrier of the BBB, astrocytes, pericytes and other neurons are responsible for reacting to signals from the capillaries and executing downstream events which add further complexity to the BBB.

While the assembly of TJs are critical for the formation of an effective barrier, assessing the integrity of the BBB goes well beyond simply detecting occludin or claudin protein expression at the cell surface. Multiple means of investigation are required to fully assess the functionality of the BBB. A given set of parameters have been proposed to ideally address in vitro models of the BBB. An ideal BBB model would express both TJ and AJ proteins, allow limited permeability based on both molecular weight, allow selective permeability to certain ions, and express membrane transporters as well as the appropriate cell surface receptors for cell-meditated internalization (Banerjee, Shi et al. 2016).

The most widely used method to assign a quantitative value to barrier quality is called the transendothelial electrical resistance (TEER) value. This is an estimate of the resistance to ion (Na⁺ and Cl⁻ diffusion across the BMEC membrane; the higher the observed TEER value the ‘tighter’ the BMEC barrier, with lower values representing poor, underdeveloped or insufficient BBB functionality. While, in vivo human TEER measurements are clearly not able to be executed, approximate values have been established with values >1000 Ωcm² considered excellent. However, in vitro models typically do not approach those values; minimum values of 150-200 Ωcm² are considered acceptable for studies addressing drug permeability (Smith and Rapoport 1986; Butt, Jones et al. 1990; Reichel, Begley et al. 2003). The main difficulty in obtaining values that approach in vivo levels is the ability to harvest and maintain suitable BMEC cultures long-term. Typically cells used for in vitro studies are immortalized versions of brain microvascular cells which often do not fully represent the characteristics of freshly purified endothelial cell cultures and lack sufficient expression of TJ genes. A new approach, while technically challenging, uses differentiated induced pluripotent stem (iPS) cells to, at least temporarily, obtain TEER values >1000 Ωcm² in vitro, a significant improvement (Lippmann, Azarin et al. 2012; Lippmann, Al-Ahmad et al. 2014).

Another important evaluation of BBB functionality is permeability. Paracellular transport of tracer dyes can provide information on the ability of small hydrophilic molecules to cross the BMEC monolayer. Materials such as FITC-labeled dextrans, sucrose, or lucifer-yellow dyes can be used to establish an endothelial permeability coefficient (Pₑ). For example, the tiny disaccharide sucrose (molecular weight 342 g/mol) has a Pₑ 0.03×10⁻⁶ cm/s⁻¹. Higher observed Pₑ would values indicate a more permeable BBB (Bickel 2005; Czupalla, Liebner et al. 2014; Banerjee, Shi et al. 2016). While TEER and Pₑ are directly correlated for smaller hydrophilic molecules, molecular weight and charge play critical roles as well; therefore tracer dyes with differing molecular weights are also useful for establishing confidence in observed Pₑ values.

Currently in vitro models employ either freshly-derived (primary) BMEC typically obtained from rodent or bovine sources (Banerjee, Shi et al. 2016) or immortalized BMEC, typically human. Freshly-derived BMEC provide superior TEER and permeability values when compared to their immortalized counterpart cultures, though they have a finite lifespan and limited population doublings making long-term studies difficult to perform. Alternatively, established cell lines generated by immortalizing normal human BMEC-derived from autopsy patients are often used for in vitro studies. Yet, these immortalized cultures such as HBEC-5i or hCMEC/D3 cells have poor TEER values, often <50 Ωcm² and especially poor Pₑ values ranging from 10-50×10⁻⁶ cm/s⁻' (Banerjee, Shi et al. 2016). As mentioned earlier, improved methods for deriving BMEC using iPS cells have been established cells and have greatly improved TEER and Pₑ values for up to 50 hrs in culture, though significant reductions are observed thereafter (Lippmann, Azarin et al. 2012). Nevertheless, iPS cells offer the best opportunity to develop improved in vitro models for which to assess the BBB.

Widely used approaches to the in vitro BBB model use mono, co- or even tri-culture conditions to establish a functional BBB using transwell membrane well plates. In such cases, a monolayer of BMEC are grown on a porous transwell cell culture membrane and other cell types such as astrocytes and/or pericytes are added to the model, in various arrangements, in order to better recapitulate the in vivo BBB (Banerjee, Shi et al. 2016; Herland, van der Meer et al. 2016; van der Helm, van der Meer et al. 2016). However, drawbacks and shortcomings to this model are extensive. For example, TEER measurements are expected to be the resistance calculated across a single monolayer of BMEC, however BMEC plated on one side of the porous transwell plates have been observed to migrate to the opposing side of the membrane establishing a duplicate monolayer. This double layer of BMEC significantly impacts the TEER values collected and also disrupts endothelial cell polarity required for proper BBB function (Wuest and Lee 2012; Vandenhaute, Drolez et al. 2016). Similarly, astrocytes are glial cells which are morphologically very similar to other neurons, in that they send out foot processes (i.e. cellular appendages) that come in close proximity to BMEC. Yet, in the current in vitro models BMEC are grown on one side of the porous transwell membrane while astrocytes are placed on the other side. Shayan et al have shown foot processes extending toward BMEC traverse the pores of the transwell membrane to reach the BMEC, however in so doing the foot processes themselves actually block the pores of the transwell membrane and limit the amount of soluble factors secreted by the astrocyte from reaching the endothelial cell, and this in turn significantly impacts the properties of the BBB (Shayan, Choi et al. 2011).

Other more recent approaches have been reported including organ-on-chip technology to assess BBB functionality. Organ-on-chip devices employ microfluidics which permit the introduction of perfusion, a critical element which has been found to improve not only TEER values but also Pe measurements. A significant advantage to the organ-on-chip approach is the ability to apply shear forces through perfusion which more accurately represent the in vivo state, whereby physiologically relevant blood pressure and intracranial pressures can be applied (van der Helm, van der Meer et al. 2016). Yet, these models are also not without limitations. Blood vessel mimics currently in use poorly represent the physical dimensions of human brain capillaries with luminal and abluminal diameters of 2 and 5 mm respectively, which are values nearly an order of magnitude larger than typical brain capillary and can in turn can affect shear forces. Further, models of this diameter require significant numbers of BMEC in order to fill the luminal space which makes creating long stretches of microvessel nearly impossible to generate using primary BMEC due to limited availability and limited doubling capacities. Furthermore, these organ-on-chip constructs are typically constructed using polydimethylsiloxane (PDMS) microchannels separated by polycarbonate (PC) membranes integrated into the device. Disadvantages to this method include the inability to manipulate the microvessel as it is fixed in place within the device. Furthermore, the PDMS/PC microchannel approach is not a biologically responsive material and does not support endothelial sprouting (the outgrowth of endothelial cells), a critical feature of in vivo brain blood vessel development.

A need exists for improved techniques to model the blood-brain barrier.

### BRIEF SUMMARY

Synthetic human blood vessels can be constructed using human brain derived endothelial cells and incorporated into a tissue model that contains astrocytes and other neurons and microglia. Multi-cell type microvessels incorporate cell types such as astrocytes and pericytes in order to construct a highly representative blood-brain barrier in vitro model with a functional lumen containing brain-derived microvascular endothelial cells and a polymer wall containing human astrocytes and/or pericytes. A microfluidic method based on sheath flow generates hollow microvessels that can incorporate cells present in the blood brain barrier in order to provide a superior blood brain barrier model and eliminate the need for unreliable transwell membrane-based assays.

In one embodiment, a synthetic blood vessel includes a hollow tube having a lumen and a polymer wall comprising extracellular matrix (ECM) components, the tube having an outer diameter of 50 μm to 250 μm and living brain microvascular endothelial cells (BMEC) disposed within the lumen.

In another embodiment, a synthetic blood vessel includes a hollow tube having a lumen and a polymer wall comprising extracellular matrix (ECM) components, the tube having an outer diameter of 50 μm to 250 μm; living brain microvascular endothelial cells (BMEC) disposed within the lumen; and living astrocytes disposed within the polymer wall.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 is a schematic representation of the neural vascular unit (NVU) which comprises brain microvascular endothelial cell (BMEC), astrocytes, other neurons and pericytes. Far right, depicts a typical brain capillary with a 2-6 gm outer diameter. BMEC are linked to neighboring endothelial cells through the expression of tight junction proteins. Left, shows linkages of pericytes to the periphery of the blood vessel and astrocyte foot processes are shown extending toward the outer wall of the vessel interacting with other neurons at their opposing end. (Source: Banerjee, Shi et al. 2016).

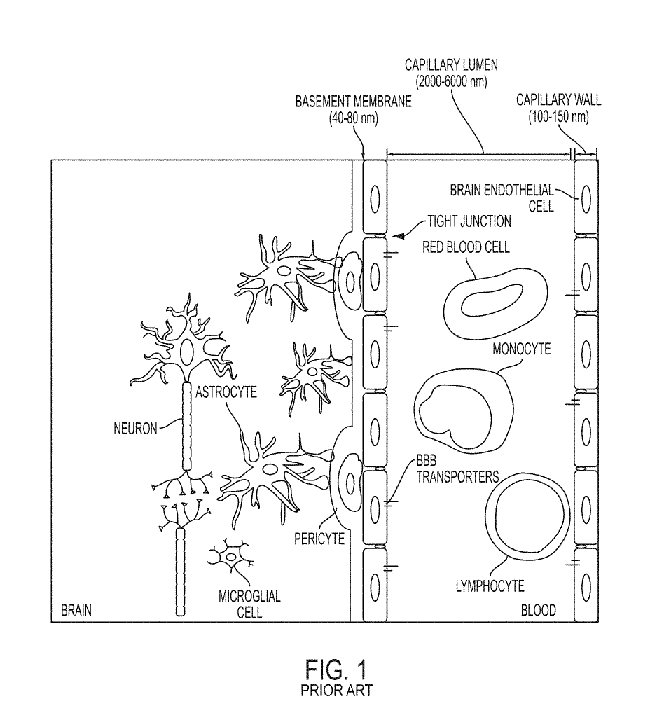

FIG. 2 depicts formation of brain microvascualr enothelial cell tight junctions. Occludin and Claudins 3 and 5 are transmembrane cell adhesion molecules which are involved in the majority of the endothelial tight junctions, while zona occludin-1 (ZO1, 2 and 3) act as intracellular linkages to the transmembrane proteins. Other cell adhesion molecules include junctional adhesion molecules (JAM), platelet endothelial cell adhesion molecule (PECAM) and the cadherins. (Source: www.bloodbrainbarrier.worldpress.com).

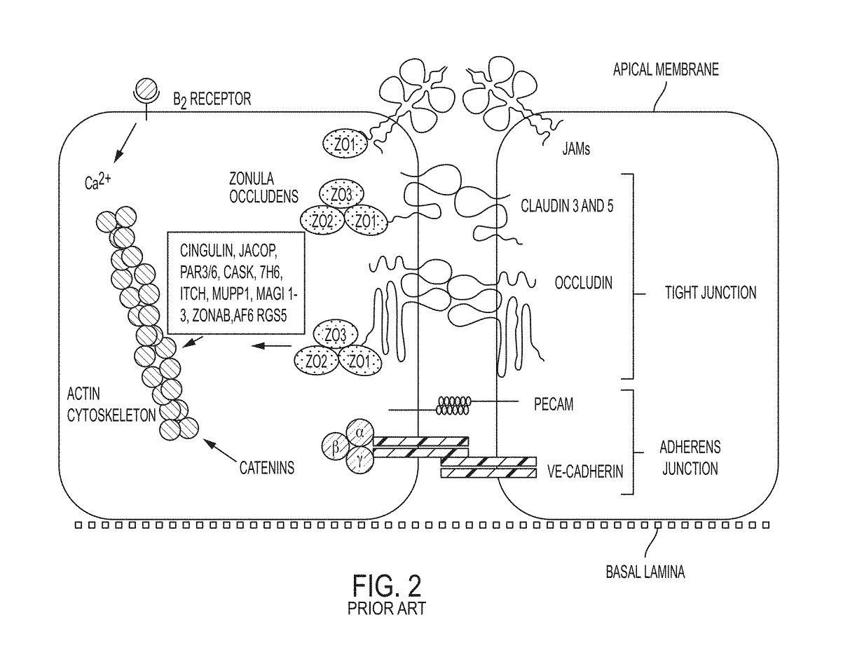

FIGS. 3A through 3D show constructed single-cell type human brain-derived endothelial microvessels (HBDEM) embedded in an extracellular matrix. A, 10× magnification of Day 7 HBDEM were placed into an extracellular matrix containing human astrocytes and image represents time zero after embedding where astrocyte outgrowth has not yet occurred. B, Represents viable HBDEM embedded in an extracellular matrix at day 7, here astrocytes are undergoing outgrowth and extending foot processes toward the HBDEM. C, 20× magnification of astrocytes interacting with outer wall of the HBDEM. D, DiL live-cell fluorescent dye (red) incorporated into astrocytes shows the position of the astrocytes with respect to the HBDEM.

FIGS. 4A-4E show multi-cell HBDEM. A, 10× transmission image shows day 10 microvessels constructed with human brain microvascular endothelial cells present in the microvessel lumen, while astrocytes are incorporated into the microvessel wall during construction. B, Shows an overlay image of DiL live-cell stained (red) astrocytes. BMEC are stained with the anti-CD31/PECAM (green) endothelial biomarker. C, overlay 10× image showing DiL astrocytes (red), anti-CD31/PECAM immuno-stained BMEC (green), and DAPI-labeled nuclei (blue). D, 20× magnification transmission image highlights extensive outgrowth of astrocytes present in the microvessel polymer wall by day 10. E, overlay image showing 20× magnification of DiL stained astrocytes (red) and anti-CD31/PECAM immuno-stained BMEC cells (green).

### DETAILED DESCRIPTION

Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

The model described herein represents a substantial improvement beyond current in vitro transwell and other organ-on-chip methodologies. It employs technology recently developed and patented (U.S. Pat. No. 9,157,060) at the U.S. Naval Research Laboratory to construct synthetic blood vessels, termed human endothelial microvessels (HEMV). Further details regarding the formation of such synthetic micro blood vessels and other fibers can be fond in U.S. Pat. Nos. 8,361,413, 8,398,935, and 9,573,311. Each of these four patents is incorporated herein by reference for the purposes of disclosing devices and methods (such as sheath flow) for preparing hollow fibers suitable for use as synthetic blood vessels.

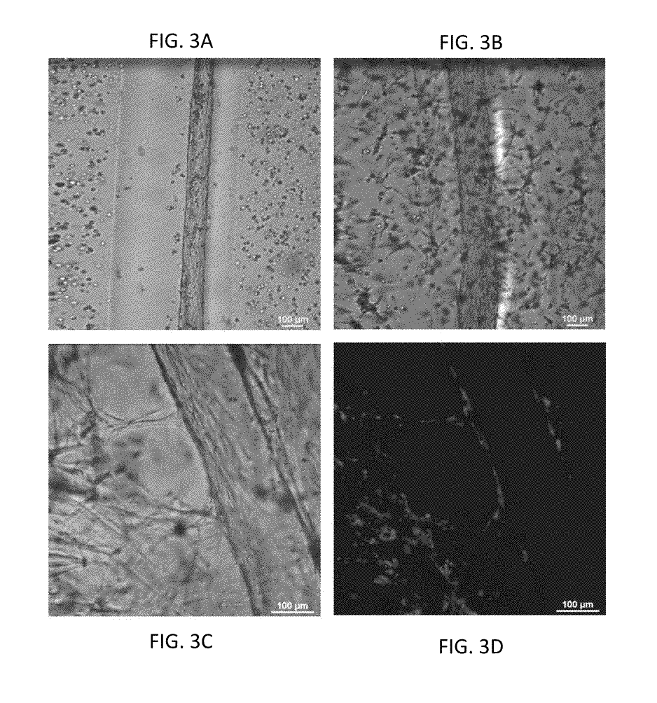

Synethic HEMV can be modified and tailored for use in addressing the blood-brain barrier in an in vitro research setting. In this model, BMEC either of primary, immortalized or iPS origin can be incorporated into the lumen of the polymer microvessel concurrently during its construction (FIGS. 3A -3D). The BMEC adhere to inner wall (luminal face) of the microvessel through the aid of extracellular matrix components such as gelatin methacrylate, fibronectin, collagen IV and hyaluronic acid, any or all of which can be included in the polymer mixture used to create the microvessels. A microvessel in this fashion, termed a human brain-derived endothelial microvessel (HBDEM) is significantly different that those developed earlier, as they are able to undergo physiologically relevant functions exclusive to brain microvessels, such expressing tight junctions and exhibiting low vascular permeability. The HBDEM are hollow by design and support perfusion of various materials including PBS, cellular growth media, simulated blood, as well as other cell types in suspension including those of the hematopoietic lineage (red and white blood cells).

The microvessel described above can recreate small, simple brain capillaries with dimensions of 50-250 μm outer diameters (OD). In embodiments, the vessel has a wall comprising one or more concentric layers of polymer, wherein the vessel has an outer diameter of between 5 and 8000 microns and wherein each individual layer of polymer has a thickness of between 0.1 and 250 microns. Yet the brain is a complex organ system that requires multi-cell interaction as described previously. In order to approximate human brain microvessels, the technique used to generate the HBDEM can be further modified by incorporating multiple cell types. The materials used to generate the polymer wall have been previously described ((Daniele, Adams et al. 2014; Daniele, Boyd et al. 2015; and U.S. Pat. No. 9,157,060 , each of which is incorporated herein by reference for the disclosure of techniques for generating appropriate components of a model tissue) though the present embodiments incorporate further steps to better recapitulate the BBB. Human astrocytes and/or human pericytes can be introduced to the polymer mixture solution and incorporated into the microvessel wall during fabrication; along with BMEC which comprise the lumen (and in embodiments exist only in the lumen), thus generating a multi-cell microvessel. The novel protocols described here allow the formation of three different types of microvessels for use in in vitro BBB analyses. The first microvessel containing BMEC without other cells to form the HBDEM (FIGS. 3A-3D) are similar, in cell type only, to mono-culture models which use BMEC in transwell membrane culture plates.

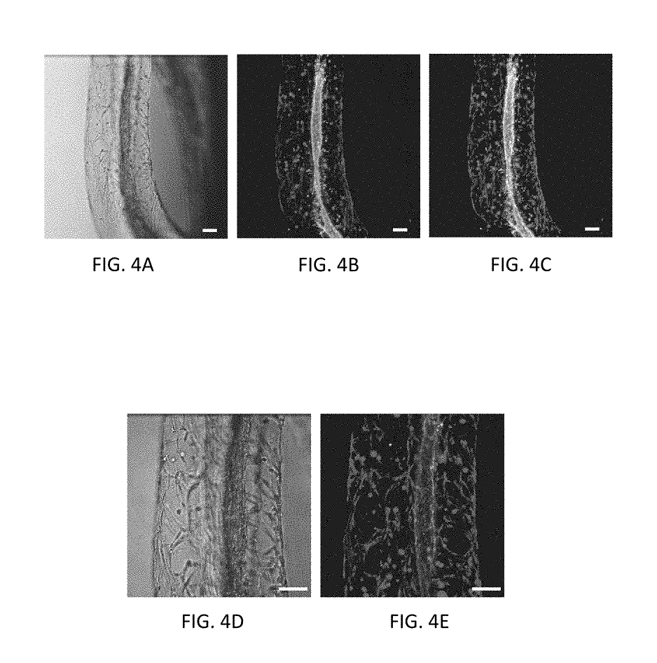

Microvessels can also be constructed using a multi-cell approach as seen in FIGS. 4A-4E. Here, astrocytes placed into the polymer wall, will begin to outgrow and interact with neighboring BMEC present in the lumen. Incorporating multiple cell types better mimics the BBB microenvironment and has been shown to stabilize and enhance TJ protein expression (Janzer and Raff 1987; Tao-Cheng, Nagy et al. 1987). Other more complex multi-cell microvessels can incorporate yet another cell type into the polymer mixture, the pericyte. A microvessel now constructed with BMEC, astrocytes and pericytes now best represents in vivo conditions present in the BBB. Using this approach, one can construct brain capillary-like microvessels which are more representative in size to observed capillaries in vivo and are capable of being positioned into any in vitro model, unlike microchannels integrated into other rigid devices. This proposed model represents an improvement over transwell-type assays which are notoriously unreliable, with users often reporting significant variability in TEER values. Furthermore, as the proposed microvessels are hollow by design the ability to perfuse material through these cell-laden microvessels vastly improves their utility, a process that is simply not possible using the transwell approach.

This model enables construction of simulated brain microvessels which incorporate all human-derived cellular components including brain microvascular endothelial cells, astrocytes and pericytes during construction of the microvessel. In contrary to other ridged devices, the constructed microvessels proposed here are freely-formed hollow tubules able to be positioned in to any in vitro device or tissue model to support tissue maintenance. Applications for these microvessels include BBB permeability studies, drug delivery research and brain-targeted diseases resulting from viral or bacterial infection. While in vivo models are the gold standard for addressing BBB functionality and drug safety, they suffer from the lack of human complementarity, with an estimated 80% of candidate drugs successfully tested in small animals failing in human clinical trials. This proposal provides a tested and validated alternative to the animal model by providing biocompatibility; an all human cellular composition; microvessels that are able to support perfusion and shear stresses; and are more comparable in size to blood vessels present in the human brain.

Further Embodiments

One of skill in the art can connect the described synthetic microvessels to equipment suitable for their use in performing desired testing. For example, such a vessel could be connected to a perfusion pump for flowing a liquid through the vessel from an inlet end thereof to an outlet end of the vessel. The liquid could contain a molecule of interest or a tracer, the presence of which could be measured as desired, e.g., in media surrounding the exterior of microvessel, as an indication of permeability.

Advantages

The engineered blood vessels described here can be free-standing and allow placement into tissue at essentially any position, unlike transwell membrane assays currently used to address blood-brain barrier functionality which use fixed monolayer cultures. Furthermore, transwell membrane assays suffer from reproducibility issues related to brain microvascular endothelial cell (BMEC) continuity.

Currently, in vitro transwell membrane assays are not suitable for perfusion. Therefore, critical elements such as shear stress forces present in vivo cannot be addressed using those models. Perfusion is a critical feature present using the proposed model.

In vivo animal models suffer from reproducibility, species complementarity and access as bans on some studies have been in place since 2013 in the European Union. Published results indicate upward of 80% of drug candidates successful in small animals fail in human clinical trials, likely due to issues related to complementarity. In contrast, the model proposed here uses an approach with all human cells

The multi-cell microvessel described herein can produce an all-human microvessel that is fully representative of brain capillaries, comprising BMEC, astrocytes and pericytes in order to best recapitulate in vivo capillary physiology.

This model is expected to aid in moving beyond current in vitro transwell membrane assays that suffer from poor reproducibility and limited options for perfusion, and make significant improvement upon other microfluidic BBB models. Compared to other microfluidic-based BBB models, the described microvessels (a) better approximate brain capillary size and critically since the proposed microvessel uses biocompatible materials; and (b) support endothelial sprouting beyond the fabricated microvessel, allowing full tissue integration and better tissue maintenance than is currently provided by other rigid microchannel devices.

Concluding Remarks

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Abbott, N. J., A. A. Patabendige, et al. (2010). “Structure and function of the blood-brain barrier.” Neurobiol Dis 37(1): 13-25.

Banerjee, J., Y. Shi, et al. (2016). “In vitro blood-brain barrier models for drug research: state-of-the-art and new perspectives on reconstituting these models on artificial basement membrane platforms.” Drug Discov Today.

Bickel, U. (2005). “How to measure drug transport across the blood-brain barrier.” NeuroRx 2(1): 15-26.

Butt, A. M., H. C. Jones, et al. (1990). “Electrical resistance across the blood-brain barrier in anaesthetized rats: a developmental study.” J Physiol 429: 47-62.

Czupalla, C. J., S. Liebner, et al. (2014). “In vitro models of the blood-brain barrier.” Methods Mol Biol 1135: 415-437.

Daniele, M. A., A. A. Adams, et al. (2014). “Interpenetrating networks based on gelatin methacrylamide and PEG formed using concurrent thiol click chemistries for hydrogel tissue engineering scaffolds.” Biomaterials 35(6): 1845-1856.

Daniele, M. A., D. A. Boyd, et al. (2015). “Microfluidic strategies for design and assembly of microfibers and nanofibers with tissue engineering and regenerative medicine applications.” Adv Healthc Mater 4(1): 11-28.

Herland, A., A. D. van der Meer, et al. (2016). “Distinct Contributions of Astrocytes and Pericytes to Neuroinflammation Identified in a 3D Human Blood-Brain Barrier on a Chip.” PLoS One 11(3): e0150360.

Janzer, R. C. and M. C. Raff (1987). “Astrocytes induce blood-brain barrier properties in endothelial cells.” Nature 325(6101): 253-257.

Lippmann, E. S., A. Al-Ahmad, et al. (2014). “A retinoic acid-enhanced, multicellular human blood-brain barrier model derived from stem cell sources.” Sci Rep 4.

Lippmann, E. S., S. M. Azarin, et al. (2012). “Derivation of blood-brain barrier endothelial cells from human pluripotent stem cells.” Nat Biotechnol 30(8): 783-791.

Reichel, A., D. J. Begley, et al. (2003). “An overview of in vitro techniques for blood-brain barrier studies.” Methods Mol Med 89: 307-324.

Satchell, S. C. and F. Braet (2009). “Glomerular endothelial cell fenestrations: an integral component of the glomerular filtration barrier.” Am J Physiol Renal Physiol 296(5): F947-956.

Shayan, G., Y. S. Choi, et al. (2011). “Murine in vitro model of the blood-brain barrier for evaluating drug transport.” European Journal of Pharmaceutical Sciences 42(1-2): 148-155.

Smith, Q. R. and S. I. Rapoport (1986). “Cerebrovascular permeability coefficients to sodium, potassium, and chloride.” J Neurochem 46(6): 1732-1742.

Sweeney, M. D., S. Ayyadurai, et al. (2016). “Pericytes of the neurovascular unit: key functions and signaling pathways.” Nat Neurosci 19(6): 771-783.

Tao-Cheng, J. H., Z. Nagy, et al. (1987). “Tight junctions of brain endothelium in vitro are enhanced by astroglia.” J Neurosci 7(10): 3293-3299.

van der Helm, M. W, A. D. van der Meer, et al. (2016). “Microfluidic organ-on-chip technology for blood-brain barrier research.” Tissue Barriers 4(1): e1142493.

Vandenhaute, E., A. Drolez, et al. (2016). “Adapting coculture in vitro models of the blood-brain barrier for use in cancer research: maintaining an appropriate endothelial monolayer for the assessment of transendothelial migration.” Lab Invest 96(5): 588-598.

Wuest, D. M. and K. H. Lee (2012). “Optimization of endothelial cell growth in a murine in vitro blood-brain barrier model.” Biotechnology Journal 7(3): 409-417.

What is claimed is:

1. A synthetic blood vessel comprising:
a hollow tube having a lumen and a polymer wall comprising extracellular matrix (ECM) components, the tube having an outer diameter of 50 μm to 250 μm and living brain microvascular endothelial cells (BMEC) disposed within the lumen.

2. The synthetic blood vessel of claim 1, further comprising living human astrocytes and/or living human pericytes.

3. The synthetic blood vessel of claim 2, wherein both living human astrocytes and living human pericytes are present.

4. The synthetic blood vessel of claim 1, wherein said ECM comprises one or more material selected from the group consisting of gelatin methacrylate, fibronectin, collagen, and hyaluronic acid.

5. The synthetic blood vessel of claim 1, wherein said BMEC are of human origin.

6. The synthetic blood vessel of claim 1, having a transendothelial electrical resistance value of >500 Ωcm².

7. A synthetic blood vessel comprising:
a hollow tube having a lumen and a polymer wall comprising extracellular matrix (ECM) components, the tube having an outer diameter of 50 μm to 250 μm;
living brain microvascular endothelial cells (BMEC) disposed within the lumen; and
living astrocytes disposed within the polymer wall.

8. The synthetic blood vessel of claim 7, further comprising living human astrocytes living human pericytes.

9. The synthetic blood vessel of claim 7, wherein said ECM comprises one or more material selected from the group consisting of gelatin methacrylate, fibronectin, collagen, and hyaluronic acid.

10. The synthetic blood vessel of claim 7, wherein the BMEC and astrocytes are of human origin.

11. The synthetic blood vessel of claim 7, having a transendothelial electrical resistance value of >500 Ωcm².
